(19) United States
(12) Patent Application Publication
(10) Pub. No.: US 2009/0257988 A1
(43) Pub. Date: Oct. 15, 2009

(54) METHODS AND COMPOSITIONS FOR ACCELERATING THE GENERATION OF REGULATORY T CELLS EX VIVO

(75) Inventors: David A. Horwitz, Santa Monica, CA (US); J. Dixon Gray, Sherman Oaks, CA (US)

Correspondence Address: MORGAN, LEWIS & BOCKIUS, LLP, ONE MARKET SPEAR STREET TOWER, SAN FRANCISCO, CA, 94105, US

(73) Assignee: USC Stevens, Hughes Center, Los Angeles, CA (US)

(21) Appl. No.: 12/421,941

(22) Filed: Apr. 10, 2009

Related U.S. Application Data
(60) Provisional application No. 61/044,306, filed on Apr. 11, 2008.

(51) Int. Cl. A61K 35/12; C12N 5/02

(52) U.S. Cl. 424/93.7; 435/375

(57) ABSTRACT

The present invention is directed to generating regulatory T cells by treating a cell culture that includes non-regulatory T cells with a regulatory composition. The invention encompasses methods utilizing a regulatory composition that includes agents that prevent methylation of the locus for the FOXP3 transcription factor, agents that accelerate differentiation of T cells into suppressor cells, and agents that are histone deacetylase inhibitors. The invention also encompasses compositions of regulatory T cells generated by culturing non-regulatory T cells with a regulatory composition as well as the use of such regulatory T cells in the treatment of autoimmune diseases and aberrant immune responses.

### CROSS REFERENCE TO RELATED APPLICATIONS

This application claims the benefit of priority to U.S. Patent Application No. 61/044,306 filed Apr. 11, 2008, which is hereby incorporated by reference in its entirety.

### STATEMENT REGARDING FEDERALLY SPONSORED RESEARCH

Not applicable.

### BACKGROUND OF THE INVENTION

Regulatory T cells (also known as “suppressor T cells” or “Tregs”) are specialized populations of T cells that act to suppress activation of the immune system and thereby maintain immune system homeostasis and tolerance to self-antigens. Regulatory T cells can occur naturally (also referred to herein as “nTregs”) or they can be induced (also referred to herein as “iTregs”) in peripheral lymphoid tissues. Induced Tregs can be generated in vivo or ex vivo, generally through stimulation of CD25-precursors in the presence of regulatory compositions. Including the cytokine TGF-β in such regulatory compositions has been shown to be effective in generating iTregs.

Although Tregs can include several T cell populations, those that express the Forkhead transcription factor (“FOXP3”) are critical for the prevention of pathologic self reactivity for maintenance of immunologic homeostasis. It has been shown that although the phenotypic properties of nTregs and iTregs are very similar (and in many cases, identical), the methylation status of the FOXP3 gene in these two populations can be different. In studies conducted on cells from mice and humans, specific regions of the FOXP3 locus have been shown to have gene methylation patterns that differ between nTregs and iTregs. In general, nTregs have regions of the FOXP3 locus that are de-methylated, whereas in iTregs, these regions are often methylated. There is a general, although not absolute, relationship between the degree of gene methylation and transcriptional activity. Some studies suggest that differences that can exist in suppressor activity between iTregs and nTregs may be due at least in part to differences in methylation patterns of the FOXP3 locus in the two types of Tregs. The acetylation status of FOXP3 is also an important determinant of its transcriptional activity.

Tregs generated ex-vivo can be divided into antigen-specific cells and polyclonal cells (polyclonal Tregs have a broad range of specificities). Both antigen-specific and polyclonal iTregs can be induced ex vivo by applying IL-2 and TGF-β to mouse cells. Polyclonal iTregs generated using IL-2 and TGF-β have been shown to have long-term beneficial effects in mouse models of systemic lupus erythematosus, autoimmune diabetes mellitus, myasthenia gravis, and allergic encephalomyelitis (reviewed in Horwitz et al., (2008), Trends Immunol., 29(9):429-35). In human cells, alloantigen iTregs have been successfully generated with IL-2, but polyclonal iTregs have been more difficult to generate ex vivo. One study has shown that although human CD4+ cells can be induced to stably express FOXP3 upon application of IL-2 and TGF-β to naïve CD4 cells, these cells failed to develop suppressive activity (Tran et al., (2007), Blood, 110(8):2983-90). However, other studies have shown that after repeated stimulation, such cells can become suppressor cells with similar characteristics of natural FOXP3+ suppressor cells (Horwitz et al., (2008), Eur J Immunol, 38(4):912-5). These cells also display membrane-bound TGF-β (another phenotypic property of nTreg suppressor cells) after repeated stimulation. Thus, although it is possible to produce iTregs with phenotypic properties that are similar to those of nTregs, conventional methods of generating iTregs usually require repeated stimulation of the cells to produce and maintain nTreg phenotypic properties and function. This failure of conventional methods utilizing TGF-β and IL-2 to generate stable suppressor cell populations without repeated stimulation, particularly in human cells, may be due at least in part to the methylation and acetylation status of the gene encoding FOXP3.

For therapeutic applications, it would be advantageous to have methods and compositions for generating therapeutic numbers of Tregs in a short amount of time without having to repeatedly stimulate T cells to induce terminal differentiation to functional suppressor cells.

### SUMMARY OF THE INVENTION

Accordingly, the present invention provides methods and compositions for generating iTregs that are phenotypically and/or functionally similar to or indistinguishable from that of nTregs.

In one aspect, the invention provides a method of generating regulatory T cells (Tregs) that includes the step of treating a cell culture that includes non-regulatory T cells with a regulatory composition. In this aspect, the regulatory composition includes an agent that prevents methylation of a gene encoding a transcription factor.

In one aspect, the invention provides a method of treating an aberrant immune response or an autoimmune disease in a patient, and this method includes the step of administering regulatory T cells to the patient. In this aspect, the regulatory T cells are generated by treatment of a cell culture that includes non-regulatory T cells with a regulatory composition. This regulatory composition may include: azacytidine, retinoic acid, trichostatin A, or a combination of two or more of azacytidine, retinoic acid and trichostatin A.

In one aspect, the invention provides a method of generating regulatory T cells (Tregs) that includes the step of treating a cell culture that includes non-regulatory T cells with a regulatory composition that includes an agent that accelerates differentiation of T cells into Tregs.

In one aspect, the invention provides a composition that includes a cell culture medium, azacytidine, retinoic acid, and a population of T cells comprising at least one naïve CD4+ cell.

In one aspect, the invention provides a kit that includes a regulatory composition, a cell treatment container, and written instructions for use of the kit. In a further aspect, the regulatory composition included in the kit includes azacytidine, retinoic acid, or a combination of azacytidine and retinoic acid.

### BRIEF DESCRIPTION OF THE DRAWINGS

FIG. 1 shows FOXP3 (abscissa) and CD25 (ordinate) expression in CD4+ cells stimulated with anti-CD3/anti-CD28 beads in the presence or absence of TGF-β.

FIG. 2 shows the effects of azacytidine, TGF-β and ALK5i on FOXP3 expression. FIG. 2A shows FOXP3 expression in cells stimulated in medium (left panel), in the presence of TGF-β (middle panel) and in the presence of azacytidine (right panel). FIG. 2B shows the percentage of cells expressing FOXP3 for cultures stimulated in medium, in medium containing ALK5i, and in the solvent DMSO.

FIG. 3 shows the additive effects of azacytidine and TGF-β on FOXP3 expression. FIG. 3A shows data from flow cytometry experiments analyzing the expression of FOXP3 in cells stimulated by anti-CD3/anti-CD28 beads in medium alone, in medium containing TGF-β, in medium containing azacytidine, and in medium containing both azacytidine and TGF-β. FIG. 3B is a bar graph of at least three separate similar experiments showing the percentage of cells expressing FOXP3 after stimulation in the presence or absence of azacytidine, TGF-β, and both azacytidine and TGF-β.

FIG. 4 is a bar graph showing the suppressive activity of cells stimulated with anti-CD3/anti-CD28 coated beads in medium alone and cells stimulated in medium containing azacytidine.

FIG. 5 shows the effects of retinoic acid on FOXP3 expression. FIG. 5A is a bar graph from cells stimulated with anti-CD3/anti-CD28 coated beads in IL-2 or IL-2 and TGF-β in different concentrations of all-trans retinoic acid. FIG. 5B shows cell counts from experiments in which naïve CD4+CD25− cells were stimulated using anti-CD3/anti-CD28 in medium alone, in the presence of TGF-β, and in the presence of TGF-β and retinoic acid.

FIG. 6 shows flow cytometry data of cells stimulated with anti-CD3/anti-CD28 coated beads in medium alone, in medium containing TGF-β, in medium containing azacytidine, in medium containing an active metabolite of retinoic acid, all trans retinoic acid (0.05 μm/ml) (ATRA), and in medium containing a combination of TGF-β, azacytidine and ATRA.

FIG. 7 shows bar graphs of CD4+ cells after six days of stimulation in medium alone, medium containing TGF-β, medium containing retinoic acid (RA), medium containing azacytidine, medium containing retinoic acid and azacytidine, and medium containing retinoic acid, azacytidine and TGF-β. The effects of these agents on expression of CD127, FOXP3, CD45RO, and CD103 are shown.

FIG. 8 shows expression of membrane-bound TGF-β in cells stimulated in medium alone (FIG. 8A), in medium containing TGF-β (FIG. 8B), and in medium containing retinoic acid, azacytidine and TGF-β (FIG. 8C). FIG. 8D shows control IgG expression in cells stimulated in medium containing retinoic acid, azacytidine and TGF-β.

FIG. 9 is a bar graph showing the increase in suppressive activity seen in cells treated with a regulatory composition containing IL-2 and TGF-β and the further increase in suppressive activity seen in cells treated with a regulatory composition containing IL-2 and TGF-β and all-trans retinoic acid (ATRA).

### DETAILED DESCRIPTION OF THE INVENTION

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3^{rd} Ed., W.H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “a polymerase” refers to one agent or mixtures of such agents, and reference to “the method” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies which are described in the publication and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

### I. Overview

The present invention is directed to methods and compositions for generating induced Tregs (“iTregs”) using a regulatory composition. Regulatory compositions of the invention can include a number of different components, as will be discussed in further detail herein. In general, the regulatory composition will include an agent that affects the methylation or acetylation of a transcription factor, an agent that affects the differentiation of T cells into suppressor cells, or a combination of such agents with other components, such as cytokines, including the cytokines TGF-β and IL-2.

The cytokines TGF-β and IL-2 are known to be sufficient to generate iTregs in mouse cells, however in human cells the use of only these cytokines may be insufficient to generate stable populations of polyclonal iTregs (although antigen-specific iTregs can be generated in human cells using IL-2 and TGF-β). Without being bound by theory, one possibility is that these cytokines induce human CD4+ cells to express and acetylate FOXP3, but further modification may be needed of the methylation and acetylation status are needed for complete maturation to functional suppressor cells. As such, the present invention includes regulatory compositions that may affect the acetylation and methylation status of FOXP3, particularly the FOXP3 gene promoter. In one embodiment the present invention includes agents that enhance acetylation of the FOXP3 gene promoter (such as retinoic acid) and/or agents that affect FOXP3 deacetylation (such as trichostatin A). As discussed herein, retinoic acid also accelerates T cell maturation into suppressor cells.

In some situations, a regulatory composition used for generating iTregs will include an agent that affects the methylation of the transcription factor FOXP3. Such an agent may be a methyltransferase inhibitor, such as azacytidine. In some situations, regulatory compositions of the invention may include an agent that accelerates T cell differentiation. Such an agent may be retinoic acid. Retinoic acid may also induce acetylation of the FOXP3 gene promoter (Kang et al., (2007) J. Immunol. 179:3724-33). Regulatory compositions of the invention may also include both an agent that affects the methylation of a transcription factor as well as an agent that accelerates T cell differentiation—i.e., regulatory compositions of the invention may include both azacytidine and retinoic acid. Other agents that enhance histone acetylation (such as trichostatin A—see Tao et al., (2007) Nat Med 13:1299-1307) may also be included in regulatory compositions of the invention. Regulatory compositions of the invention may also include cytokines, such as TGF-β and IL-2. Without being bound by theory, it is possible that any acetylating and demethylating agents included in such regulatory compositions may accelerate the differentiation and maturation of T cells induced to become Tregs.

In general, iTregs are generated in accordance with the invention by treating non-regulatory T cells with a regulatory composition. Non-regulatory T cells can include peripheral blood mononuclear cells (“PBMCs”). By “treating” is meant contacting a regulatory composition to the non-regulatory T cells, usually by applying the regulatory composition to a culture that includes the non-regulatory T cells. As will be appreciated, although cell cultures are generally discussed herein in terms of cultures of non-regulatory T cells, such cell cultures may also include other types of cells. In some situations, a regulatory composition is contacted with the cells at the initiation of the cell culture, and in some situations a regulatory composition is contacted with the cells at least once after initiation of the cell culture. In some situations a regulatory composition is contacted with the cells at the initiation of the cell culture and then again at least once after initiation of the cell culture.

Regulatory T cells generated in accordance with the present invention can be used to treat aberrant and undesirable immune responses and autoimmune diseases. In general, such regulatory T cells are introduced into a patient using methods known in the art.

The present invention also encompasses populations of iTregs generated according to methods described herein. The present invention also encompasses regulatory compositions, which can include azacytidine, retinoic acid, trichostatin A, TGF-β, IL-2 and any combination thereof. These regulatory compositions may in some situations be combined with cell culture media. In some situations, the present invention also encompasses regulatory compositions in combination with T cells.

The present invention also includes kits. Such kits may include at least one reagent, including regulatory compositions described herein for generating iTregs. Kits of the invention may also include containers for generating iTregs of the invention. Such containers may include multiple ports that allow delivery of reagents to cells within the containers. The present invention also encompasses kits for packaging and delivering iTregs to a patient. Kits of the invention may further include containers for isolating cells from patients. In some situations, kits of the invention include containers that can be used for multiple aspects of methods of the invention. For example, such containers may be adapted for isolating cells from a patient, treating the isolated cells with a regulatory composition to generate iTregs, and/or administering the newly generated iTregs to a patient.

II. Phenotypic Properties of Natural Tregs (nTregs) and Induced Tregs (iTregs)

In one aspect, the present invention provides methods and compositions that produce iTregs that have phenotypic properties of nTregs. By “phenotype” or “phenotypic property” as used herein is meant an observable characteristic. For regulatory T cells, such phenotypic properties can include without limitation: expression of certain proteins (such as cytokines and transcription factors), proliferation, and suppressor activity. For example, nTregs are known to express the transcription factor FOXP3 and can express cytokines such as transforming growth factor beta (TGF-β). nTregs tend to express only low levels of other cytokines, such as interleukin 4 (IL-4) and interleukin (IL-10). Cells displaying suppressor activity have also been shown to express the cytokine TGF-β on their membranes. Tregs with “suppressor activity” are cells with the ability to suppress proliferation and immune responses of other T cells.

A primary phenotypic property of nTregs is suppressor activity, and generation of iTregs with similar suppressor activity is one aspect of the present invention. Suppressor activity can be measured in a number of ways, including standard assays for T cell cytotoxic activity, such as inhibition of T cell proliferation, as well as assays described for example in U.S. Pat. No. 6,759,035, which is hereby incorporated by reference in its entirety for all purposes and in particular for all teachings related to assays of suppressor cell activity. Other phenotypic properties may also be detected and measured to determine if iTregs are suppressor cells and have phenotypic properties of nTregs.

One phenotypic property of nTregs is expression of the transcription factor FOXP3. FOXP3 is a master controller of nTregs and has been shown to be required for their development and function. Both mice and humans with a genetic deficiency of the FOXP3 gene develop autoimmune symptoms. Studies have shown that stimulation of murine non-regulatory T cells in the presence of the cytokines IL-2 and TGF-β results in expression of FOXP3 and the development of suppressor activity. Although FOXP3 expression is not an absolute indicator of suppressor activity, it is one phenotypic property that may be used to identify an iTreg as a suppressor cell akin to that of an nTreg.

Another phenotypic property of nTregs is expression of membrane-bound TGF-β. Detection of membrane-bound TGF-β in iTregs is thus an indication that such iTregs are suppressor cells. Methods for detection of membrane-bound TGF-β are described for example in U.S. patent application Ser. No. 12/194,101, filed on Aug. 19, 2008, which is hereby incorporated by reference in its entirety for all purposes and in particular for all teachings related to assays for membrane-bound TGF-β.

A further phenotypic property of nTregs is poor proliferative responsiveness, which is often accompanied by lowered production of certain pro-proliferation cytokines, such as IL-2. Other cytokines, such as IL-4, IFNγ and TFN-α are also associated with proliferation, although they are generally produced in low levels even in proliferating cells. Proliferation response can be measured using methods known in the art, such as thymidine uptake assays and assays of carboxyfluorescein succinimidyl ester (CFSE) dilution.

III. Generating Induced Tregs (iTregs)

A number of methods for inducing the formation of regulatory T cells exist, as described in, for example, U.S. Pat. Nos. 6,228,359; 6,358,506; 6,797,267; 6,803,036; 7,381,563 and 6,447,765, and U.S. application Ser. Nos. 10/772,768; 11/929,254; 11/400,950; and 11/394,761; all of which are hereby incorporated in their entirety for all purposes and in particular for all teachings related to the generation of regulatory T cells (“Tregs”). Although such conventional methods can produce iTregs in mice fairly rapidly, a drawback of many of these methods in human cells is that they require extended periods of time in which cultures of Treg precursors must be repeatedly stimulated in order to produce human iTregs with lasting suppressor activity. The present invention provides methods and compositions that can produce suppressor cells quickly with minimal to no need of re-stimulation during the course of generating iTregs. In general, as used herein unless otherwise indicated, to “stimulate” non-regulatory T cells means to contact the cells with one or more T cell activators, including without limitation anti-CD3 and anti-CD28. Such stimulation may be in the presence of a regulatory composition, or such stimulation may occur prior to or subsequent to contact of the non-regulatory T cells with a regulatory composition.

Regulatory Compositions

The present invention provides regulatory compositions that are of use in generating iTregs that display similar phenotypic characteristics to nTregs. By “regulatory composition” herein is meant a composition that can cause the formation of regulatory T cells from non-regulatory T cells. As is discussed in further detail below, regulatory compositions can be used to induce Tregs that have phenotypic properties similar or identical to those of nTregs. In general, regulatory compositions of the invention are added to cultures of non-regulatory T cells. Such regulatory composition may include agents that stimulate the non-regulatory T cells to differentiate into suppressor cells as well as agents that enhance that differentiation and the formation of nTreg phenotypic properties. Any of the components of the regulatory compositions described herein are also referred to as “additives”.

In general, regulatory compositions of the invention include an agent that affects the methylation of the gene for FOXP3 and/or the gene for TGF-β. The effect on gene methylation may be through direct action of the agent on the gene or indirectly through action of the agent on one or more intermediaries. In a further embodiment, the agent prevents the methylation of the FOXP3 gene and/or the gene for TGF-β. In one aspect, the agent used to prevent methylation of the FOXP3 gene is a methyltransferase inhibitor. Such methylase transferase inhibitors can for example include without limitation azacytidine (“azaC”—also known as 2′-Deoxy-5-azacytidine; 5-Aza-2′-deoxycytidine) and 1-b-D-ribofuranosyl-2(1H)-pyrimidinone.

In some aspects, regulatory compositions of the invention include agents that accelerate differentiation of T cells into suppressor cells. Such agents can include without limitation retinoic acid (particularly active metabolites of retinoic acid) and histone deacetylase inhibitors such as trichostatin A. Such agents may be used with other additives in regulatory compositions of the invention, such as cytokines and/or optionally T cell activators. Agents such as retinoic acid and trichostatin A may also be used in combination with agents that affect the methylation of transcription factors, such as azacytidine.

In addition to agents such as azacytidine, regulatory compositions of the present invention may further include cytokines such as TGF-β, IL-2, IL-7, IL-15 and TNFA, individually or in any combination.

By “transforming growth factor-β” or “TGF-β” herein is meant any one of the family of the TGF-βs, including the three isoforms TGF-β1, TGF-β2, and TGF-β3; see Massague, J. (1980), J. Ann. Rev. Cell Biol 6:597. Lymphocytes and monocytes produce the β1 isoform of this cytokine (Kehrl, J. H. et al. (1991), Int J Cell Cloning 9:438-450). The TFG-β can be any form of TFG-β that is active on the mammalian cells being treated. In humans, recombinant TFG-β is currently preferred. In general, the concentration of TGF-β used in regulatory compositions of the invention can range from about 2 pg/ml of cell suspension to about 50 ng/ml. In further embodiments, the concentration of TGF-β used in regulatory compositions of the invention ranges from about 5 pg/ml to about 40 ng/ml, from about 10 pg/ml to about 30 ng/ml, from about 20 pg/ml to about 20 ng/ml, from about 30 pg/ml to about 10 ng/ml, from about 50 pg/ml to about 1 ng/ml, from about 60 pg/ml to about 500 pg/ml, from about 70 pg/ml to about 300 pg/ml, from about 80 pg/ml to about 200 pg/ml, and from about 90 pg/ml to about 100 pg/ml. In further embodiments, the concentration of TGF-β used is determined based upon endpoints such as percentage of FOXP3+ cells produced in a population of cells and stability of FOXP3 expression. Such endpoints can be determined using methods known in the art and described herein.

IL-2 can be any form of IL-2 that is active on the mammalian cells being treated. For human cells, recombinant IL-2 is generally used. Recombinant human IL-2 can be purchased from R & D Systems (Minneapolis, Minn.). In general, the concentration of IL-2 used ranges from about 1 Unit/ml of cell suspension to about 200 U/ml. In further embodiments, the concentration of IL-2 ranges from about 1 U/ml to about 175 U/ml, from about 2 U/ml to about 150 U/ml, from about 3 U/ml to about 125 U/ml, from about 4 U/ml to about 100 U/ml, from about 5 U/ml to about 80 U/ml, from about 10 U/ml to about 70 U/ml, from about 15 U/ml to about 60 U/ml, from about 20 U/ml to about 40 U/ml, and from about 25 U/ml to about 30 U/ml.

Regulatory compositions of the invention may also include T cell activators such as anti-CD2, including anti-CD2 antibodies and the CD2 ligand, anti-CD3, anti-CD28, LFA-3, Concanavalin A (Con A), and staphylococcus enterotoxin B (SEB). In some embodiments, T cell activators are used in concentrations from about 0.1 to about 5.0 pg/ml. In further embodiments, concentrations of T cell activators range from about 0.2 to about 4.0, about 0.3 to about 3.0, about 0.4 to about 2.0, and about 0.5 to about 1.0 μg/ml. In many embodiments, anti-CD3 and anti-CD28 are used alone or in combination with TGF-β. In further embodiments, one or more other cytokines are used in combination with agents such as azacytidine and retinoic acid as well as T cell activators such as anti-CD3 and anti-CD28.

In some embodiments, regulatory compositions of the invention comprise only a single element among the agents discussed above. For example, regulatory compositions may comprise only TGF-β, only retinoic acid, only azacytidine, only trichostatin A, or only a T cell activator.

In further embodiments, regulatory compositions of the invention comprise one or more of the above agents. As will be appreciated, any combination of the agents discussed above can be included in a regulatory composition of the present invention.

In one exemplary embodiment, a regulatory composition of the invention includes azacytidine, retinoic acid, or a combination of azacytidine and retinoic acid. Such a regulatory composition may in a further embodiment include one or more cytokines. For example, such a regulatory composition may further include TGF-β, IL-2, or both TGF-β and IL-2. Such a regulatory composition may also in a further embodiment include one or more T cell activators, such as anti-CD3 and anti-CD28.

In one exemplary embodiment, a regulatory composition of the invention includes azacytidine, trichostatin A, or a combination of azacytidine and trichostatin A. Such a regulatory composition may in a further embodiment include one or more cytokines. For example, such a regulatory composition may further include TGF-β, IL-2, or both TGF-β and IL-2. Such a regulatory composition may also in a further embodiment include one or more T cell activators, such as anti-CD3 and anti-CD28.

In one exemplary embodiment, a regulatory composition includes azacytidine and TGF-β. In a further embodiment, a regulatory composition also includes azacytidine, retinoic acid and TGF-β. In a further embodiment, such a regulatory composition also includes IL-2. In a still further embodiment, such a regulatory composition also includes at least one T cell activator such as anti-CD3 and/or anti-CD28.

In one embodiment, a regulatory composition of the invention includes azacytidine, retinoic acid, trichostatin A, and IL-2.

In one exemplary embodiment, a regulatory composition of the invention includes azacytidine and retinoic acid. In a further embodiment, the regulatory composition also includes T cell activators such as anti-CD3 and anti-CD28. In some embodiments, T cell activators are provided in the regulatory composition on beads, whereas the azacytidine and the retinoic acid are present in solution. In a still further embodiment, the regulatory composition also includes TGF-β.

In one exemplary embodiment, a regulatory composition of the invention includes azacytidine, retinoic acid, IL-2 and TGF-β. In a further embodiment, these elements of the regulatory composition are contained in a cell culture medium.

In one exemplary embodiment, a regulatory composition of the invention includes azacytidine, trichostatin A, IL-2 and TGF-β. In a further embodiment, these elements of the regulatory composition are contained in a cell culture medium.

In one exemplary embodiment, a regulatory composition includes IL-2 and TGF-β. In a further embodiment, such a regulatory composition also includes an agent to accelerate differentiation of T cells into suppressor T cells—such an agent may for example include retinoic acid. In a still further embodiment, such a regulatory composition also includes an agent that promotes demethylation, such as azacytidine. In a still further embodiment, such a regulatory composition also includes an agent that enhances histone acetylation, such as trichostatin A and/or retinoic acid.

In an exemplary embodiment, agents included in regulatory compositions of the invention have an additive or synergistic effect. For example, the use of trichostatin A and retinoic acid in a regulatory composition may have an additive or synergistic effect resulting in a larger number generated iTregs than is seen by using either agent alone. Such a synergistic/additive effect may in part be due to such agents having separate mechanisms of action on histone acetylation that together result in increased numbers of iTregs. In a further exemplary embodiment, any combination of agents described herein, including TGF-β, IL-2, azacytidine, retinoic acid and trichostatin A may have synergistic or additive effects in the generation of iTregs.

Treating Cultures of Non-Regulatory T Cells

In one aspect, the present invention provides methods for generating therapeutic numbers of iTregs within about seven to about ten days. This relatively short amount of time for generating iTregs offers an advantage over methods in the art used to expand naturally occurring Tregs (nTregs). Expansion of nTregs generally requires at least three weeks to expand a population of isolated nTregs to therapeutic numbers. This amount of time is necessary in part because only it is generally only possible to isolate a small population of nTregs, so each cell in the population must undergo a large number of cell divisions to generate a therapeutic number of cells. So many cell divisions can affect the overall suppressor activity and other phenotypic properties of the resultant population of cells. So many divisions may also alter the proliferative ability of these cells following transfer into a patient (for example, for treatment of an undesirable or aberrant immune response or autoimmune disease) and decrease their survival in vivo. Since the population of cells used to generate iTregs in accordance with the present invention is generally larger than is possible to obtain from isolation of nTregs, therapeutic numbers of cells can be generated without requiring each cell to divide as many times as is necessary when expanding smaller populations of cells.

In one aspect, the present invention provides methods of treating non-regulatory T cells with a regulatory composition to induce regulatory T cells (iTregs). As used herein, non-regulatory T cells include T cells that can be induced to have regulatory activity. Such cells include peripheral blood mononuclear cells (PBMCs), which can include primarily naïve CD-4+, CD-8+ cells, and may possibly include Natural Killer (NK) cells, and Natural Killer T (NKT) cells.

The iTregs generated using methods and compositions of the present invention will generally have suppressor activity and phenotypic properties that are similar or identical to those of naturally occurring Tregs (nTregs). By “treating” herein is meant that the cells are contacted with the regulatory composition. In an exemplary embodiment, treating the cells includes incubating the cells with the regulatory composition (for example by adding regulatory composition to the cell culture medium) for a time period sufficient for the cells to develop phenotypic properties and functions of nTregs. The incubation is generally conducted under physiological temperature.

In general, iTregs generated using methods and compositions of the present invention involve T cell receptor stimulation by one or more T cell activators. Such T cell activators can include anti-CD3, anti-CD28, anti-CD2, and combinations thereof. Such T cell activators may be included in the regulatory composition, or they may be applied to the non-regulatory T cells separately, prior to or simultaneously with a regulatory compositions of the invention. In some embodiments, the non-regulatory T cells may be “primed”, i.e., contacted with, one or more components of a regulatory composition prior to stimulation with a T cell activator.

In one aspect, treating cultures of non-regulatory T cells with any of the regulatory compositions described herein generates iTregs in a much shorter time than is possible using other methods known in the art. In one aspect, the methods and compositions of the present invention generate iTregs from non-regulatory T cell cultures within a week. In a further aspect, the methods and compositions of the present invention generate iTregs from non-regulatory T cell cultures over a period of about five days to about fifteen days, of about six days to about twelve days, and of about seven days to about ten days. In a still further aspect, the generation of iTregs does not require repeated stimulation with T cell activators such as anti-CD3 and anti-CD28. As will be appreciated, repeated stimulation can be used and is encompassed by the present invention, but is not always necessary with the regulatory compositions described herein.

Although an aspect of the invention is to generate iTregs in a shorter time period than is possible using conventional methods known in the art, the present invention also encompasses methods that generate iTregs over a longer period of time. In an exemplary aspect, methods and compositions of the present invention generate iTregs from non-regulatory T cell cultures over a period of about three days to about four weeks. In still further aspects, the methods and compositions of the present invention generate iTregs from non-regulatory T cell cultures over a period of about five days to about three weeks, from about seven days to about fifteen days, and from about ten days to about twelve days. As will be appreciated, a wide range of culture times and conditions are encompassed by the present invention. A cell culture may be maintained for purposes of the present invention before and after addition of regulatory compositions as described herein for about 2 days to about 3 months, for about 3 days to about 2 months, for about 4 days to about 1 month, for about 5 days to about 20 days, for about 6 days to about 15 days, for about 7 days to about 10 days, and for about 8 days to about 9 days

In one embodiment of the invention, a regulatory composition of the invention is contacted with non-regulatory T cells at the initiation of a culture of the cells. In another embodiment, a regulatory composition is contacted with the cells at a later time point after initiation of the culture. In a further embodiment, a regulatory composition is contacted with the cells at the initiation of a culture and at a later time point. The later time point for the first or subsequent contact of the regulatory composition can be in the range of 0.5 hour to 5 days after initiation of the culture. In another embodiment, the later time point for the first or subsequent addition of a regulatory composition can be in the range of about 1 hour to about 3 days, about 2 hours to about 2 days, about 3 hours to about 36 hours, about 4 hours to about 24 hours, about 5 hours to about 20 hours, about 6 hours to about 15 hours, and about 7 hours to about 10 hours after initiation of the culture. As discussed herein, such regulatory compositions can include azacytidine alone or in combination with one or more cytokines (including without limitation TGF-β and IL-2) as well as agents such as retinoic acid and/or trichostatin A.

In one aspect, endogenous TGF-β upregulated by application of azacytidine is used to generate Tregs. In another aspect, exogenous TGF-β is added along with azacytidine to a culture to induce FOXP3 expression. In one embodiment, TGF-β and azacytidine are added to the culture simultaneously.

In another embodiment, TGF-β and azacytidine are added to the culture sequentially—either TGF-β or azacytidine can be added first. In still another embodiment, TGF-β and azacytidine are added to the culture at different time points. In yet another embodiment, TGF-β and azacytidine, whether they are added simultaneously, sequentially or at separate time points, are applied to the culture two or more times during the lifetime of the cell culture. In further embodiments, agents that affect differentiation of T cells, such as retinoic acid, may be added simultaneously with or sequentially with other agents described herein, including without limitation azacytidine and TGF-β.

In further embodiments, different regulatory compositions and/or components of regulatory compositions are contacted with cells at different time points during the culture. In one exemplary embodiment, a regulatory composition is contacted with the cells at the initiation of the culture and at least once more during the lifetime of the culture. In a further embodiment, a regulatory composition is contacted with the cells at the initiation of the culture, and one or more components of the regulatory composition are again contacted with the cells at least once more during the lifetime of the culture. For example, a regulatory composition comprising azacytidine, TGF-β and retinoic acid is contacted with non-regulatory T cells at the initiation of a culture, and then azacytidine, TGF-β or retinoic acid is added again at least once more during the lifetime of the culture. In a further exemplary embodiment, some combination of azacytidine, TGF-β and/or retinoic acid is added at least once more during the lifetime of the culture. Similarly, for an exemplary regulatory composition comprising TGF-β, IL-2, and one or more demethylating agents and histone deacetylase inhibitors, the full regulatory composition may be added at one time point and one or more components may additionally be contacted with the culture at subsequent time points alone or in combination with other components of the regulatory composition or with other additives, including cytokines, T cell activators, as well as fresh cell culture media and/or other agents known to affect the health and stability of cell cultures. As will be appreciated, any combination of components of a regulatory composition can be added at one or more time points during the lifetime of a cell culture.

In some embodiments, one or more components of a regulatory composition are added to a culture of cells at least once after initiation of the culture. In further embodiments, one or more components of a regulatory composition are added to a culture of cells from about 2 to about 15 times during the lifetime of the culture. In still further embodiments, one or more components of a regulatory composition are added to a culture of cells from about 3 to about 14, about 4 to about 13, about 5 to about 12, about 6 to about 11, about 7 to about 10, and about 8 to about 9 times during the lifetime of a culture. In these embodiments, the culture of cells may comprise non-regulatory T cells, regulatory T cells, and both non-regulatory and regulatory T cells. The regulatory T cells may be nTregs and/or iTregs. These cultures may also include cells other than T cells.

In further exemplary embodiments, one or more agents are contacted with cultures of non-regulatory T cells sequentially or simultaneously. For example, in embodiments in which azacytidine and retinoic acid are used to generate iTregs, the azacytidine and retinoic acid may be contacted with the cells simultaneously with retinoic acid or sequentially in any order. Similarly, in embodiments in which azacytidine, retinoic acid, and TGF-β are used to generate iTregs, the three agents can be contacted with the non-regulatory cells simultaneously or sequentially in order. As will be appreciated, any of the combinations of agents described herein that can be included in regulatory compositions can be contacted with cells simultaneously or sequentially in any order.

In one aspect, the invention provides a method of generating regulatory T cells (Tregs) that includes the step of treating a culture of non-regulatory T cells with a regulatory composition. In this aspect, the regulatory composition includes an agent that prevents methylation of a gene encoding a transcription factor. In an exemplary embodiment, the agent that prevents methylation of a gene encoding a transcription factor is a methyltransferase inhibitor that prevents methylation of the gene for FOXP3. In a further exemplary embodiment, the methyltransferase inhibitor is azacytidine. In a still further embodiment, the regulatory composition also includes a cytokine, such as TGF-β. In a yet further embodiment, the regulatory composition also includes an agent that accelerates T cell differentiation, such as retinoic acid. In a still further embodiment, the regulatory composition includes a histone deacetylase inhibitor, such as trichostatin A. As will be appreciated, any combination of a subset of these components of this exemplary regulatory composition may be used to generate iTregs.

In one embodiment, treating the culture of non-regulatory T cells includes adding the regulatory composition at the initiation of the culture. In one embodiment, the treating of the culture of non-regulatory T cells includes adding the regulatory composition after initiation of the culture. In some embodiments, the culture of the non-regulatory T cells is maintained for about one week after the treating with the regulatory composition, whether that treating occurs at the initiation of the culture or subsequent to the initiation of the culture.

In a further embodiment, an agent is added to the culture of non-regulatory T cells at a second time point subsequent to the addition of the regulatory composition at the initiation of the culture. In a still further embodiment, the agent is added multiple times during the lifetime of the culture. In such embodiments, the agent added one or more times after initiation of a culture may be azacytidine, retinoic acid, trichostatin A, TGF-β, IL-2, anti-CD3, anti-CD28, or some combination of these or any other components of regulatory compositions described herein.

In some embodiments, prior to treatment with a regulatory composition and prior to stimulation with a T cell activator, the non-regulatory T cells are “primed” with one or more agents. By “primed” is meant that the non-regulatory T cells are contacted with the one or more agents prior to contact with the regulatory composition. For example, the cells may be contacted with azacytidine, retinoic acid, trichostatin A, TGF-β, IL-2 or some combination thereof prior to initiation of cell culture and/or prior to contact with a regulatory composition that may contain one or more of the agents used to prime the cells. In an exemplary embodiment, cultures of non-regulatory T cells are primed with TGF-β and IL-2, stimulated with a T cell activator in the presence of a regulatory composition comprising an agent that affects the methylation of FOXP3, an agent that affects the differentiation of cells into suppressor cells, an agent that is a histone deacetylase inhibitor, or some combination of the three agents. In further exemplary embodiments, the regulatory composition comprises azacytidine, retinoic acid, trichostatin A, or some combination of the three. In still further embodiments, the regulatory composition also includes TGF-β and/or IL-2.

In some embodiments, prior to treatment with a regulatory composition, non-regulatory T cells may be subjected to one or more pre-treatment protocols. For example, once collected, the cells can be additionally concentrated using standard techniques in the art, including without limitation use of Ficoll-Hypaque density gradient centrifugation.

In a further embodiment, after one or more concentration steps, the cells can be washed to remove serum proteins and soluble blood components, such as autoantibodies, inhibitors, etc., using techniques well known in the art. Generally, such techniques involve addition of physiological media or buffer, followed by centrifugation. Such steps may be repeated as necessary.

After one or more rounds of concentration and/or purification, the cells may in further embodiments be resuspended in physiological media, such as AIM-V serum free medium (Life Technologies) or buffers such as Hanks balanced salt solution (HBBS) or physiological buffered saline (PBS) can also be used. If physiological media are used, serum free media are preferred, as serum can otherwise contain proteins that act as inhibitors of iTreg generation.

In one embodiment, the cells may be enriched for one or more cell types prior to treatment with a regulatory composition. For example, the cells may be enriched for CD8+ T cells or CD4+ T cells using techniques well known in the art, such as through the use of commercially immunoabsorbent columns as well as other techniques, such as those described in Gray et al. (1998), J. Immunol. 160:2248, which hereby incorporated by reference in its entirety for all purposes and in particular for all teachings related to enriching a population of cells for one or more cell types.

In another embodiment, the PBMCs are separated in an automated, closed system such as the Nexell Isolex 300i Magnetic Cell Selection System, a Miltenyi “AutoMACS system” or a flow cytometers. In general, such separation is conducted using methods and devices known in the art to maintain sterility and ensure standardization of the methodology used for cell separation, activation and development of suppressor cell function. In many embodiments, once the cells have undergone any necessary pretreatment, the cells are treated with a regulatory composition.

In one embodiment, non-regulatory T cells are collected using leukopheresis collection methods, resulting in a concentrated sample of cells in a sterile leukopak. In a still further embodiment, the leukopak may be modified for the addition of reagents and/or doses of the regulatory composition as a kit, such that treatment of the cells to generate iTregs can take place in the same leukopak into which the sample was collected. Such kits are discussed in more detail below.

In some aspects, the regulatory compositions and the methods described herein are used to expand naturally occurring Tregs (nTregs) as well as to induce regulatory T cells from non-regulatory T cells. In some embodiments, expanding nTregs will utilize regulatory compositions described herein that include IL-2. In further embodiments, expanding nTregs using methods described herein comprise treating a population of isolated nTregs with a regulatory composition. This regulatory composition will in further embodiments include IL-2. In still further embodiments, the nTregs are treated with IL-2 and one or more additional agents and/or cytokines, including TGF-β, azacytidine, retinoic acid, and trichostatin A. Any of the methods described above for treating non-regulatory T cells to generate iTregs are also applicable to expanding populations of nTregs.

As will be appreciated, the present invention in one aspect includes compositions of iTregs generated according to the methods described herein.

In addition, the present invention also includes compositions that include cell culture medium, an agent that affects methylation of a transcription factor (such as azacytidine), an agent that affects differentiation of T cells into suppressor cells (such as retinoic acid), and a population of T cells comprising at least one naïve CD4+ cell. Such a composition may also include at least one induced regulatory T cell. In a further embodiment, the at least one induced regulatory T cell is a suppressor T cell. In a still further embodiment, the composition further includes a histone deacetylase inhibitor such as trichostatin A. T cell activators may in some embodiments be added to this composition of the invention to further induce iTregs.

In one aspect, the invention includes compositions that include cell culture medium, an agent that affects methylation of a transcription factor (such as azacytidine), an agent that affects differentiation of T cells into suppressor cells (such as retinoic acid), and a population of T cells comprising at least one natural Treg. Such a composition may be further treated with one or more T cell activators as well as other additives, such as a histone deacetylase inhibitor or a cytokine such as IL-2 to expand the nTregs. As will be appreciated, this expanded population of nTregs is also encompassed by the present invention.

Assessing iTregs for nTreq Phenotypic Properties

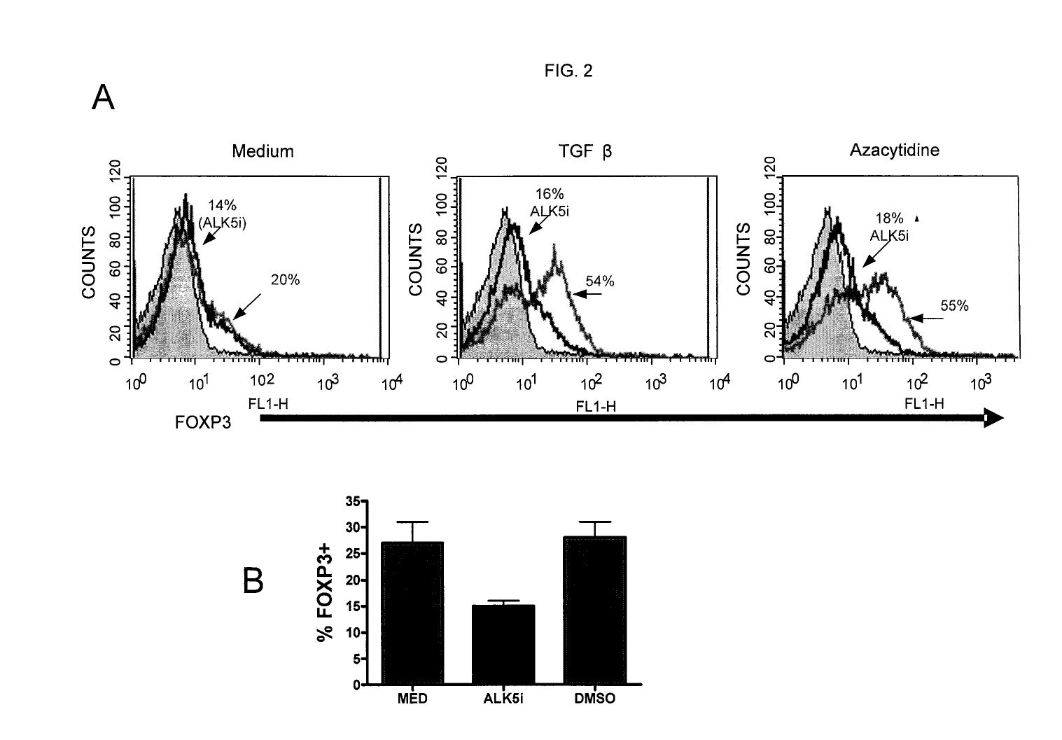

In one aspect, the methods and compositions of the present invention include treating a culture of non-regulatory T cells with a regulatory composition comprising azacytidine to stimulate iTregs and augment the percentage of FOXP3 positive cells in the culture. FIG. 2 shows that in the absence of exogenous TGF-β, stimulation of non-regulatory T cell cultures with azacytidine markedly augments the percentage of FOXP3 positive cells (the arrows in FIG. 2A indicate the augmentation due to either TGF-β (middle panel) or azacytidine (right-most panel)). As shown in FIG. 2A, this augmentation in the percentage of FOXP3+ cells may be at least partially dependent on a mechanism involving endogenous TGF-β, even when the augmentation is induced by azacytidine alone, because ALK5i inhibited FOXP3 expression induced by azacytidine to the same extent as FOXP3 expression induced by TGF-β (see traces indicated by arrows labeled “ALK5i”). The shaded areas in the graphs in FIG. 2A indicate background staining.

“Azacytidine-generated iTregs” of the invention include those generated using azacytidine alone as well as those generated using azacytidine in combination with other agents, including without limitation cytokines (such as TGF-β and IL-2) and agents that promote differentiation of T cells into suppressor cells (such as retinoic acid).

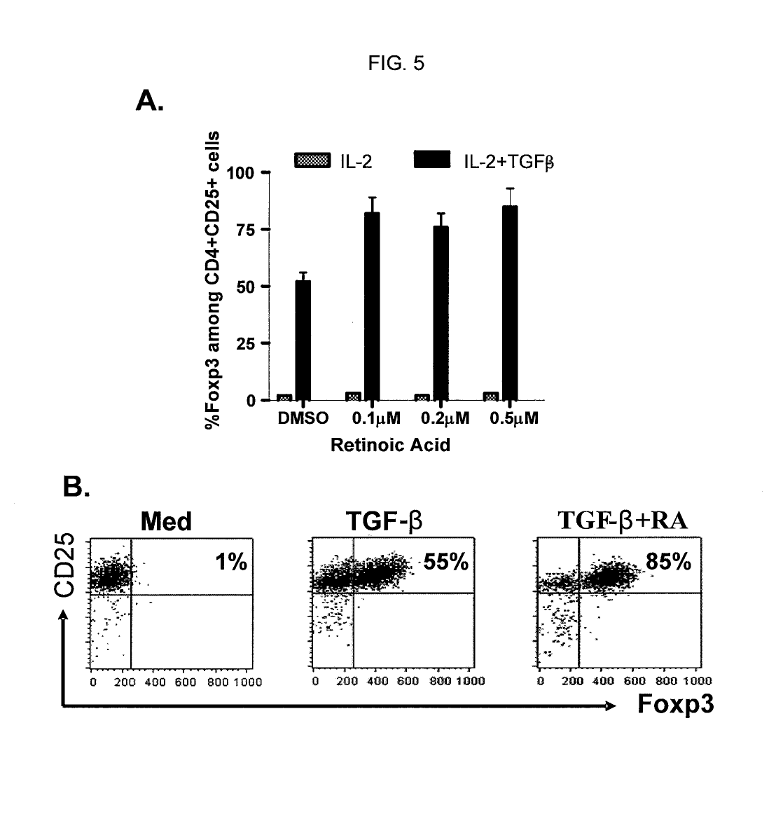

As discussed above, one phenotypic property of nTregs is poor proliferative responsiveness. Human naïve CD4+ cells stimulated with IL-2 and TGF-β become FOXP3+, but are only partially differentiated suppressor cells. FIG. 5A shows that such iTregs will proliferate when restimulated. In contrast, CD4+ cells cultured with azacytidine become non-responsive after 6 days of culture and fail to proliferate upon re-stimulation. Since the only source of pro-proliferative cytokines are the iTregs themselves, the lack of proliferation by azacytidine-treated cells is consistent with poor cytokine production, which is a distinguishing characteristic of nTregs. The addition of azacytidine combined with TGF-β also decreases the proliferative activity of these cells.

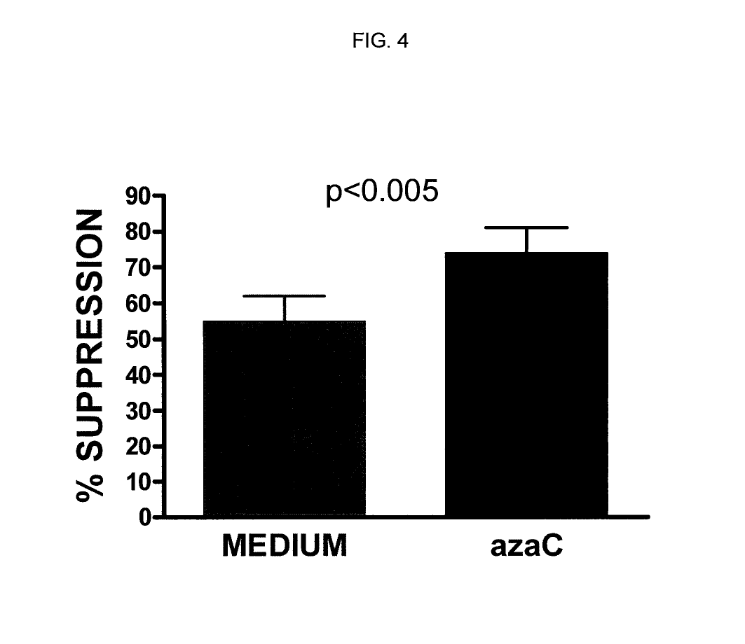

In one aspect, iTregs generated in accordance with the present invention are assessed for suppressor activity. When assayed for suppressor activity, human cells cultured in the presence of azacytidine are significantly more suppressive than cells cultured in the absence of azacytidine (FIG. 4). Thus, culture in the presence of azacytidine in accordance with the present invention is consistent with the ability to promote the generation of iTregs cells with the same or similar phenotypic and functional characteristics as nTreg.

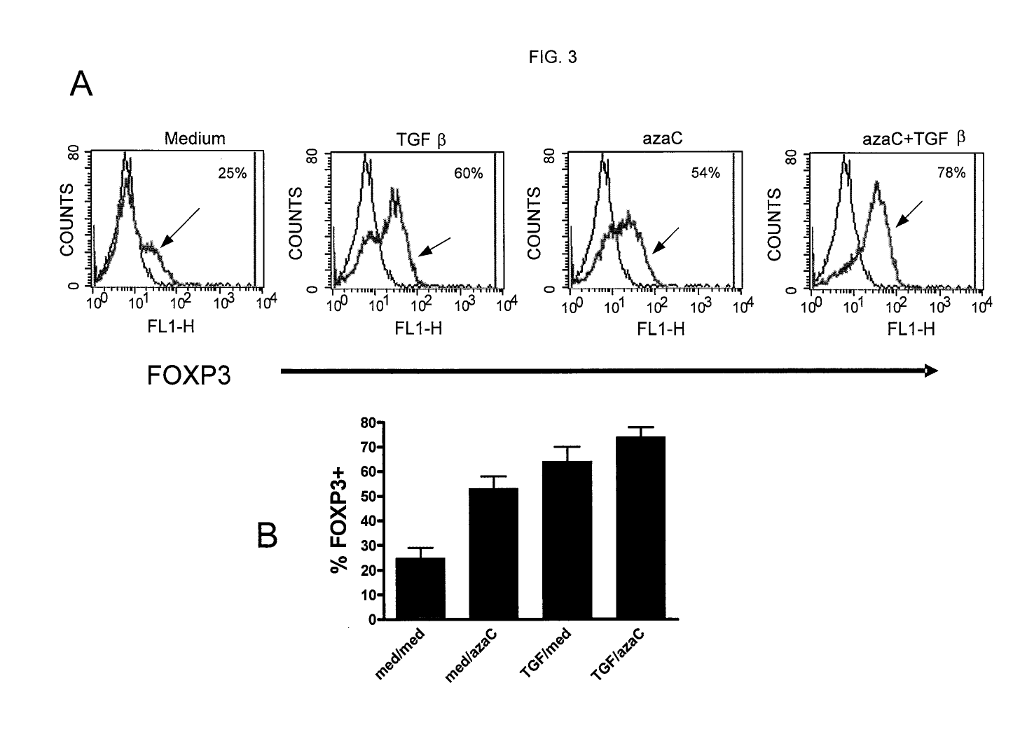

In one aspect, TGF-β is added along with azacytidine to a culture of non-regulatory T cells to induce FOXP3 expression. FIG. 3A shows that the combination of azacytidine and TGF-β has an additive effect and induces a higher percentage of FOXP3+ cells than either agent added alone.

As discussed above, in one embodiment, TGF-β and azacytidine are added to the culture simultaneously. In another embodiment, TGF-β and azacytidine are added to the culture sequentially—either TGF-β or azacytidine can be added first. In still another embodiment, TGF-β and azacytidine are added to the culture at different time points. In yet another embodiment, TGF-β and azacytidine, whether they are added simultaneously, sequentially or at separate time points, are applied to the culture two or more times during the lifetime of the cell culture.

Retinoic acid, a metabolite of Vitamin A, has been found to enable antigen presenting cells in the gastrointestinal tract to induce CD4+ cells to become FOXP3+iTregs through a TGF-β dependent mechanism. (see e.g., Kang et al., (2007) J. Immunol., 179:3724-3733. Since one of the major effects of retinoic acid is to accelerate cell maturation, the inventors reasoned that retinoic acid by itself or in combination with azacytidine may also enhance the differentiation of human iTregs. As such, the present invention encompasses methods and compositions utilizing regulatory compositions containing retinoic acid alone or in combination with any of the agents and compositions described herein, including cytokines such as IL-2 and TGF-β, T cell activators, and agents that affect methylation of transcription factors, such as azacytidine.

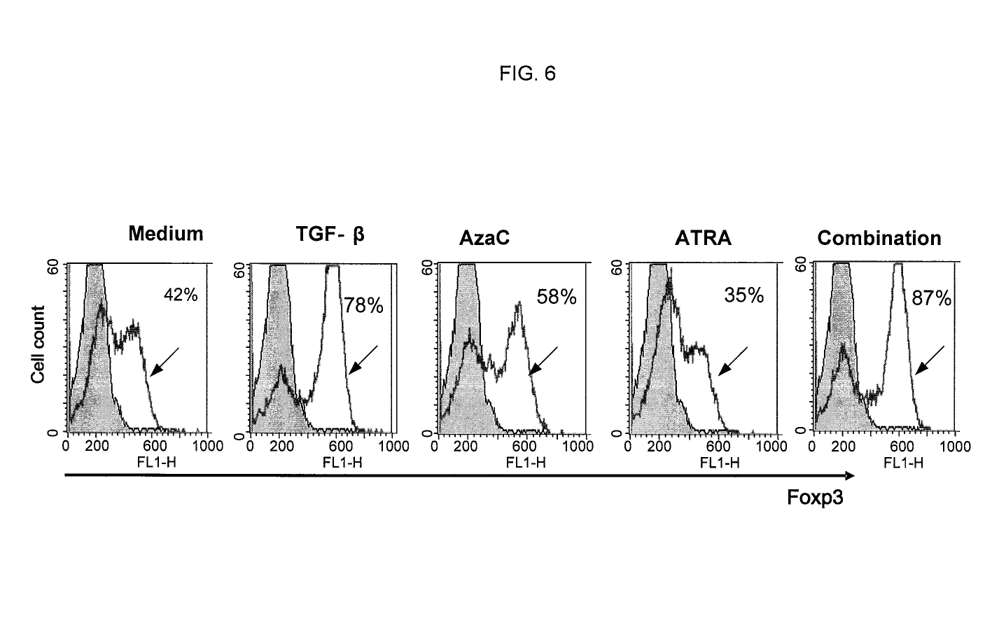

FIG. 6 demonstrates that the combination of IL-2, TGF-β, ATRA (all trans retinoic acid, which is an active metabolite of retinoic acid) and azacytidine induce a higher percentage of naïve CD4+ cells to become FOXP3+ cells than is seen by application of any of the agents by themselves. The traces in FIG. 6 indicated by the arrows are the cell counts after stimulation with anti-CD3/anti-CD28 beads.

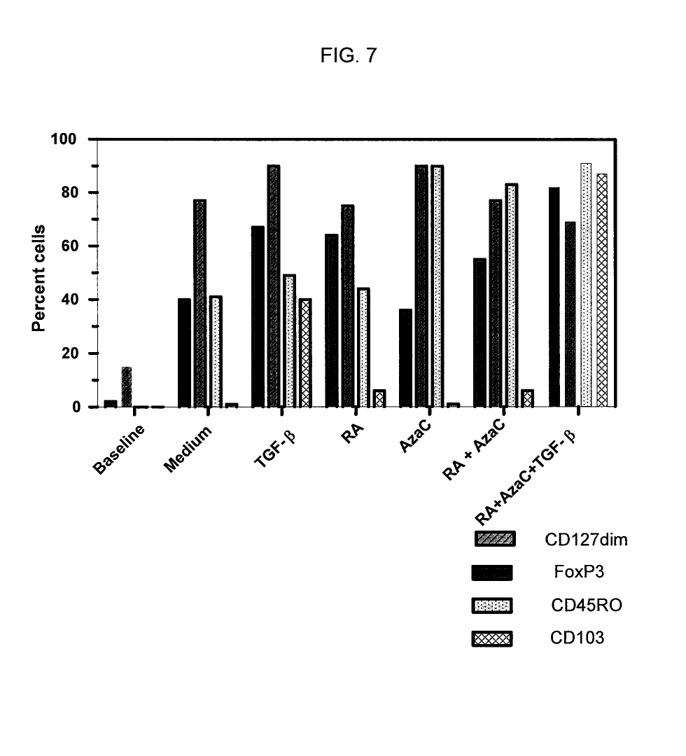

As discussed above, in one exemplary embodiment, iTregs are generated through treatment of non-regulatory T cells with a combination of retinoic acid, azacytidine, IL-2 and TGF-β. Such iTregs can be assessed for nTreg phenotypic properties, such as specific surface markers that are characteristic of mature FOXP3+nTregs. Naïve T cells display the CD45RA+ marker and lack CD45RO. After activation when they displayed the memory phenotype, these cells became CD45RA-CD45RO+. (FIG. 7) After 6 days of stimulation with TGF-β, only 50% acquired the CD45RO marker (FIG. 7). However, when azacytidine and retinoic acid were included in the cultures, almost all the cells became CD45RO+. Like nTregs, the naïve CD4+ cells showed markedly diminished expression of the IL-7 receptor (CD127) and became CD127dim. Finally, nTregs characteristically express the αEβ7 integrin (CD103) induced by TGF-β. The addition of azacytidine and retinoic to TGF-β markedly increased the percentage of CD4+ cells that expressed CD103. The combination of TGF-β, azacytidine and retinoic acid also induced naïve human CD4+ cells to express membrane-bound TGF-β. Although some T cells primed with TGF-β now expressed this cytokine on their cell surface after re-stimulation, this number was doubled if they were also primed with azacytidine and retinoic acid.

IV. Using iTregs of the Invention

The present invention encompasses populations of iTregs generated using methods and compositions described herein. Such populations of iTregs can be used in therapeutic and research applications.

In one aspect, Tregs induced using methods and compositions described herein are administered to patients suffering from, for example, aberrant immune responses and/or autoimmune diseases. In a further aspect, Tregs induced using methods and compositions described herein can be used to prevent or treat allograft rejection.

Tregs induced using methods and compositions of the invention can be administered to patients using methods generally known in the art. Such methods include without limitation injecting or introducing the iTregs into a patient. In some embodiments, iTregs are introduced into a patient via intravenous administration. In further embodiments, additional reagents such as buffers, salts or other pharmaceutically acceptable additives may be administered in combination with iTregs.

After introducing the cells into the patient, the effect of the treatment may be evaluated using methods known in the art. Examples of such evaluations can include without limitation: measuring titers of total Ior of specific immunoglobulins, renal function tests, tissue damage evaluation, and the like.

Treatment using Tregs of the invention may be repeated as needed or required. For example, the treatment may be done once a week for a period of weeks, or multiple times a week for a period of time, for example 3-5 times over a two week period. Over time, the patient may experience a relapse of symptoms, at which point the treatments may be repeated.

In one exemplary aspect, the invention provides a method of treating an aberrant immune response or an autoimmune disease in a patient, and this method includes the step of administering regulatory T cells to the patient. In this aspect, the regulatory T cells are generated by treatment of a culture of non-regulatory T cells with a regulatory composition. This regulatory composition may include: azacytidine, retinoic acid, trichostatin A, or a combination of two or more of azacytidine, retinoic acid and trichostatin A.

In one embodiment, the regulatory T cells administered to a patient are generated using a regulatory composition comprising one or more of azacytidine, retinoic acid, trichostatin A. In a further embodiment, the regulatory composition may include TGF-β and/or IL-2. In a still further embodiment, the regulatory composition may also include a T cell activator, including without limitation anti-CD3, anti-CD28, or a combination of anti-CD3 and anti-CD28.

In a further exemplary embodiment, the regulatory T cells administered to a patient for treatment of an aberrant immune response are generated from a culture of non regulatory T cells, where that culture of non-regulatory T cells is stimulated with a T cell activator prior to, simultaneously with, or subsequent to the treatment with a regulatory composition.

### V. Kits

In one aspect, the present invention provides kits for generating iTregs. In general, such kits include a sterile closed system that allows treatment of non-regulatory cells with regulatory compositions described herein without requiring the use of specialized cell treatment facilities.

In an exemplary embodiment, a kit of the invention includes a cell treatment container. The cell treatment container will in many embodiments be a closed sterile system in which non-regulatory T cells can be treated with a regulatory composition without risk of contamination. The form and composition of the cell treatment container may vary, as will be appreciated by those in the art. Generally, the container may be in a number of different forms, including a flexible bag, similar to an IV bag, or a rigid container similar to a cell culture vessel. Generally, the composition of the container will be any suitable, biologically inert material, such as glass or plastic, including polypropylene, polyethylene, etc.

In a further embodiment, the cell treatment container comprises one or more ports such that reagents for the generation of Tregs can be introduced to cells within the cell treatment container without disturbing the reaction conditions necessary to maintain the cells growing in culture. For example, a cell treatment container of the invention may include one port for the introduction of fresh cell culture medium, whereas another port is used to introduce components of regulatory compositions described herein, such as azacytidine, retinoic acid, and one or more cytokines (including without limitation TGF-β and IL-2). As will be appreciated, a wide range of designs for ports into such cell treatment containers are known in the art and are encompassed by the present invention.

In a still further embodiment, a cell treatment container of the invention will include components that can be used for separation of cells, such that only T cells remain in the container for treatment with a regulatory composition. For example, antibodies can be introduced into the container through a dedicated port or through a port that is also used to introduce other agents and molecules into the system. Such antibodies can be specific for non-T cells, such that those non-T cells can be identified and then removed from the container, leaving only T cells for treatment with other components included with the kit. In one exemplary embodiment, immunomagnetic beads are added to the cell treatment container to bind non-T cells labeled with antibodies, and those immunomagnetic beads can then be removed from the container using methods known in the art.

In one aspect, the present invention provides kits for administering iTregs to a patient. In a further aspect, kits for administering iTregs to a patient are combined with some or all components of kits for generating iTregs. Such kits may in some exemplary embodiments include cell treatment containers, such as those described above, comprising multiple ports for addition of regulatory compositions to non-regulatory T cells to generate iTregs. Such cell treatment containers may further include additional compartments and/or ports such that the iTregs can then be administered to a patient using methods known in the art, such as through intravenous (I.V.) transfusion. For example, the cell treatment containers described above may further comprise a port that is adapted for connection to an I.V. bag for administration of iTregs to a patient.

In a further aspect, kits of the invention may include cell treatment containers that can also be used during collection of cells from a patient. In one exemplary embodiment, a kit of the invention includes a cell treatment container that is adapted to be attached to a leukopheresis machine using an inlet port, such that the same container can be used for both gathering the cells and then for treating the cells to generate iTregs. In a further exemplary embodiment, the container may include further adaptations that allow it to be used to administer the generated iTregs to a patient, for example, through an adapter to an I.V. setup, as discussed above.

In further embodiments, kits of the invention may include a separate cell collection container that can be used to collect the cells from a patient, and those cells are then introduced to a cell treatment container, which is also a part of the kit. That cell treatment container may further include adaptations that allow regulatory compositions to be introduced to the cells in the container to generate iTregs, and the resultant iTregs may be administered to a patient from the cell treatment container, or the iTregs may be transferred to a separate cell administration container, which may also be included in the kit. The iTregs could then be administered to the patient from the cell administration container.

In further embodiments, kits of the invention may include cell collection containers that include elements for cell separation and purification, such that separation and/or purification of non-regulatory T cells can be conducted in the same container as is the treatment with a regulatory composition. In still further embodiments, cells are removed from the cell collection container for separation and/or purification, and after such separation and/or purification, the cells are introduced into the cell treatment container. Containers and reagents for such separation and/or purification outside of the cell collection container may also be included within the same kit.

Kits of the invention may further include at least one dose of a regulatory composition. “Dose” in this context means an amount of the regulatory composition that is sufficient to cause an effect. In further embodiments, multiple doses of a regulatory composition may be included in kits of the invention. In still further embodiments, the dose(s) of regulatory composition may be added to the cell treatment container using a port; alternatively, in some embodiments, the dose is already present in the cell treatment container. In still further embodiments, the dose(s) of regulatory composition is in a lyophilized form, which can be reconstituted using cell media or other reagents known in the art.

In further embodiments, kits of the invention may include buffers, salts, media, proteins, drugs, and other components known in the art that can be used in combination with regulatory compositions described herein to generate iTregs. Such components may also further be used as part of kits used for administering iTregs to patients.

In further embodiments, materials or components assembled in a kit of the invention can be provided to the practitioner and stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. The packaging materials employed in the kit are those customarily utilized in laboratory kits. As used herein, the term “package” refers to a suitable solid matrix or material such as glass, plastic, paper, foil, and the like, capable of holding the individual kit components. Thus, for example, a package can be a glass vial used to contain suitable quantities of a regulatory composition. The packaging material generally has an external label which indicates the contents and/or purpose of the kit and/or its components.

In still further embodiments, kits of the invention may additionally comprise written instructions for using the kits.

In some exemplary embodiments of the invention in which the kits comprise elements for cell separation, the kits contain GMP quality biotinylated antibodies. Such antibodies can include without limitation: anti-CD14 to remove monocytes, anti-CD11b or CD56 to remove NK cells, and CD8 to remove CD8 cells. Such kits could further contain magnetic beads with avidin-bound anti-mouse IgG to remove the stained monocytes, B cells, and NK cells. In still further embodiments, such kits would also include regulatory compositions, including regulatory compositions comprising azacytidine, retinoic acid, trichostatin A, TGF-β, IL-2, as well as regulatory compositions comprising combinations of two or more of these components.

In some embodiments, kits of the invention will include a cell treatment container and a regulatory composition. In an exemplary embodiment, the regulatory composition will include azacytidine, retinoic acid and TGF-β. In a further exemplary embodiment, the regulatory composition will also include IL-2. In a still further exemplary embodiment, such kits will include one or more T cell activators and cytokines, either in separate containers or as part of the regulatory composition. In still further embodiments, such kits will also include buffers, drugs, and cell culture media. In some embodiments, such kits may additionally comprise written instructions for using the kits.

In one exemplary aspect, the invention provides a kit that includes a regulatory composition, a cell treatment container, and written instructions for use of the kit. In a further aspect, the regulatory composition included in the kit includes azacytidine, retinoic acid, or a combination of azacytidine and retinoic acid. In one embodiment, the regulatory composition may further include TGF-β, IL-2, or a combination of TGF-β and IL-2. In a further embodiment, the regulatory composition further includes trichostatin A. In a still further embodiment, the cell treatment container of this exemplary kit includes a port adapted for attachment to a leukopheresis machine.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

### FOXP3 Expression in Stimulated CD4+ Cells

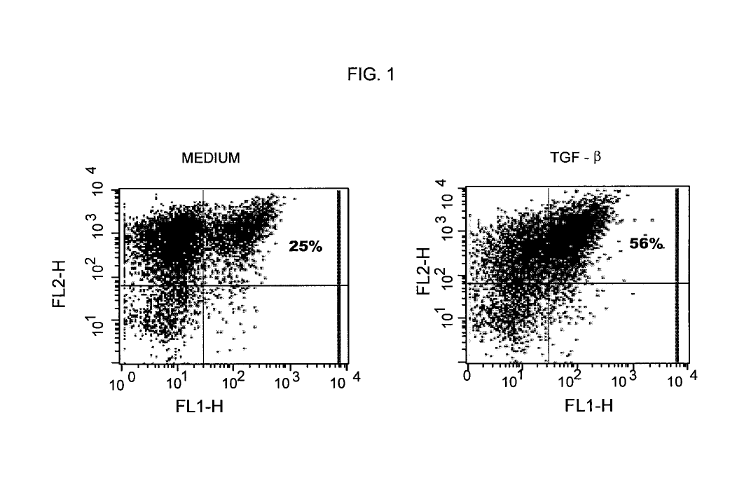

FIG. 1 is a typical example of FOXP3 expression at day 6 of cultures of non-regulatory T cells. In the experiments represented in this figure, naïve CD4+ cells were stimulated with 3/28 beads (1 bead per 10 T cells) with (right panel) or without (left panel) TGF-β for 6 days. The data show that FOXP3 expression was enhanced by TGF-β (the percentage of FOXP3 expressing cells is indicated in each graph). While FOXP3 is expressed by cells from both cultures, there are twice as many cells which express FOXP3 from the cultures which had TGF-β added.

### Example 2

### Comparison of Azacytidine and TGF-β Effect on FOXP3 Expression

FIG. 2A shows the effect of activin receptor-like kinase 5 inhibitor (ALK5i) on FOXP3 expression. This inhibitor blocks TGF-β type I receptor signaling. The figures in FIG. 2A provide data from flow cytometry analysis of stimulated CD4+ cells. Naïve CD4+ cells were stimulated with anti-CD3/CD28 beads for 5 or 6 days in medium only (left-most panel), in the presence of 5 ng/ml TGF-β (middle panel) or with 1 μM azacytidine (right-most panel). The graphs also show data from cells stimulated with and without ALK5i (10 μM). The shaded area shows cells stained with control IgG only. FIG. 2A shows that azacytidine enhances FOXP3 expression to a similar extent as the enhancement seen with TGF-β. The data in FIG. 2A also suggests that the enhancement of FOXP3 expression by azacytidine is at least partially TGF-β dependent, because the ALK5i inhibited FOXP3 expression induced by azacytidine to the same extent as FOXP3 expression induced by TGF-β.

FIG. 2B shows the mean±SEM of 5 experiments measuring the percentage of FOXP3 expression after stimulation in medium, in medium containing an ALK5i inhibitor, and in solvent only (DMSO). The figure shows that even background FOXP3 expression by stimulated CD4+ cells may be partially TGF-β dependent, because the addition of ALK5i to cells stimulated in medium was able to reduce FOXP3 expression.

### Example 3

### Assessment of Additive Effects of TGF-β and Azacytidine on FOXP3 Expression

The additive effects of TGF-β and azacytidine on FOXP3 expression were analyzed. FIG. 3 shows data demonstrating the additive effects of TGF-β and azacytidine on FOXP3 expression. FIG. 3A provides flow cytometry data from naïve CD4 cells stimulated with anti-CD3/CD28 beads for 5 or 6 days in (in order of the panels from left to right) medium alone, in medium containing TGF-β, in medium containing azacytidine, and in medium containing azacytidine and TGF-β. The arrows indicate the cell counts after stimulation with anti-CD3/anti-CD28. FIG. 3B shows the mean±SEM of 5 experiments and demonstrates the percentage of FOXP3 expression after stimulation in medium only, in medium containing azacytidine (1 μM), in medium containing TGF-β (5 ng/ml), and in medium containing both azacytidine and TGF-β. These data suggest that azacytidine and TGF-β have an additive effect in enhancing FOXP3 expression.

### Example 4

### Assessment of Suppressive Activity of Cells Stimulated in the Presence of Azacytidine

FIG. 4 demonstrates that CD4+ cells stimulated with azacytidine develop suppressive activity. The figure shows the mean±SEM of 4 experiments where naïve CD4+ cells were stimulated with anti-CD3/CD28 beads in medium alone and in medium containing azacytidine. Each population of stimulated cells was assayed for suppressive activity by culturing the cells with responder T cells labeled with carbofluorescein diacetate succinimydil ester (CFSE) at a ratio of 1:10. The cells were stimulated with soluble anti-CD3 for 3 days and proliferation of the responder cells was measured by dilution of CFSE. These data show that the enhancement of FOXP3 expression seen in populations of cells stimulated in the presence of azacytidine (see FIG. 3) is accompanied by an enhancement in the suppressive activity of these cells.

### Example 5

### Assessment of the Effect of Retinoic Acid on FOXP3+Expression

The addition of retinoic acid to TGF-β and azacytidine enhances FOXP3 expression in stimulated CD4+ cells. Naïve CD4+ cells were stimulated with anti-CD3/28 beads as described above. FIG. 5 shows data from cells stimulated in the presence of IL-2 (20 u/ml)±TGF-β (2 ng/ml)±all-trans retinoic acid (“ATRA”) (0.1-0.5 μM) or DMSO for 4 days. FOXP3 expression among CD4+CD25+ cells was determined by flow cytometry. FIG. 5A is a bar graph showing the indicated mean±SEM for 5 independent experiments. FIG. 5B is representative of experiments in 0.1 μM ATRA.

FIG. 6 shows further flow cytometry data of cells stimulated in medium alone, in medium containing TGF-β, in medium containing azacytidine, in medium containing an active metabolite of retinoic acid, all trans retinoic acid (0.05 μm/ml) (ATRA), and in medium containing a combination of TGF-β, azacytidine and ATRA. The traces identified with arrows indicate data from cells expressing FOXP3 after stimulation, and percentage of FOXP3 expressing cells is indicated in each figure. Clearly, the combination of TGF-β, azacytidine and ATRA had the most significant effect on enhancing FOXP3 expression.

### Example 6

### Assessment of the Effect of Retinoic Acid on Phenotype of Tregs

The combination of TGF-β, azacytidine and retinoic acid increases CD4+ cells with the phenotype of mature FOXP3+Treg cells. At baseline, naïve CD4+ cells do not express FOXP3, CD103, or CD45RO. Such cells also stain brightly for CD127. Following stimulation with suboptimal numbers of anti-CD3/28 beads, 70 to 85% of naïve CD4+ cells express FOXP3, CD45RO and CD103 and also show dim staining for CD127. (FIG. 7). These are all markers of mature FOXP3 CD4+ Treg cells.

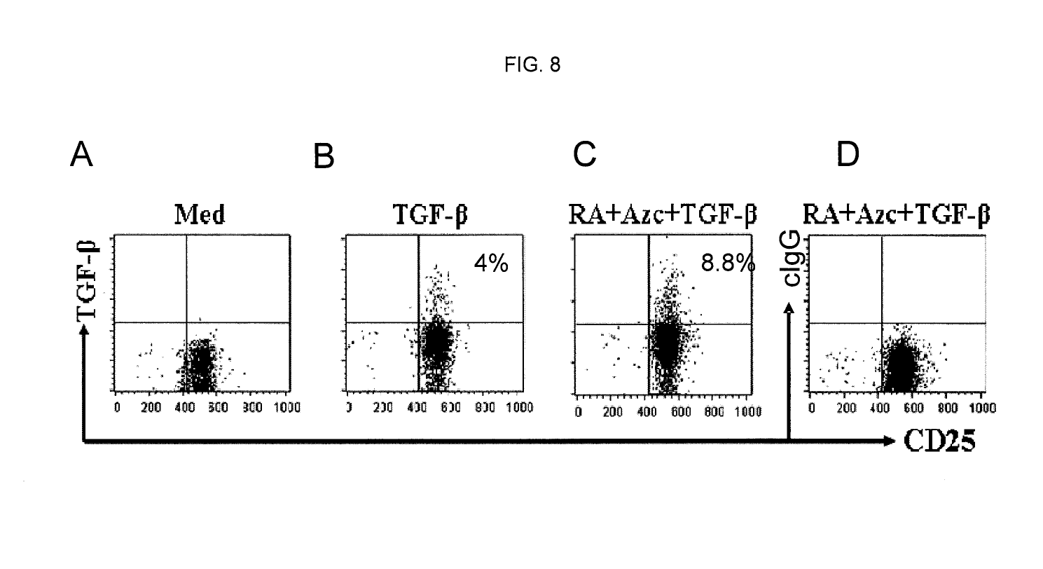

The combination of TGF-β, azacytidine and retinoic acid can induce naïve CD4+ cells to express membrane-bound TGF-β. Naïve CD4+ cells were stimulated with agents indicated for 6 days and re-stimulated with anti-CD3/28 beads and stained with fluorochrome-conjugated anti-TGF-β for membrane-bound TGF-β. FIG. 8 shows that some T cells contacted with TGF-β prior to stimulation now expressed this cytokine on their cell surface (FIG. 8B) and this number was doubled if in cells contacted with azacytidine and retinoic acid (FIG. 8C). The data in FIG. 8D show control data for the expression of IgG.

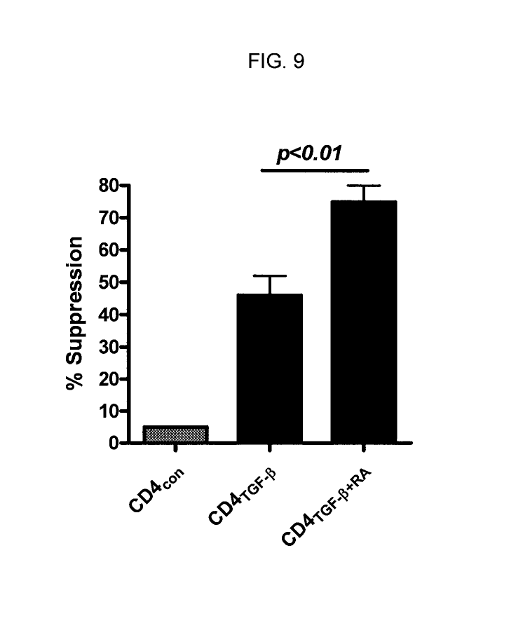

FIG. 9 shows that naïve CD4+CD25− cells stimulated in the presence of a combination of IL-2 (20 U/ml), TGF-β (2 ng/ml) and all-trans retinoic acid (0.1 μM) for 4 days showed increased suppression over cells stimulated in the presence of TGF-β alone or in IL-2 alone (CD4-con). The induced Tregs were added to T respond cells (1:4 ratio) and suppressive activity was calculated.

### Example 7

### Stimulation of Memory CD4+ Cells in the Presence and Absence of Azacytidine

In addition to naïve CD4+ cells (also referred to as CD45RA+ cells), memory CD4+ cells (CD45RO+) are stimulated and assessed for the effect of azacytidine and azacytidine+retinoic acid on their phenotypic properties. These memory CD4+ cells represent a resting but previously activated population. The cells are stimulated with anti-CD3/anti-CD28 beads (at 1 in 10) with or without azacytidine (1 μM), azacytidine combined with all-trans retinoic acid and also with or without TGF-β (5 ng/ml). After 6 days, the cells are depleted of stimulating beads and assayed for FOXP3 expression, suppressor activity and proliferative activity. Greater than 50% of the stimulated cells express FOXP3. Enhanced FOXP3 expression in cells cultured in medium alone may be due to TGF-β provided by non-T cells in the culture. Cells treated with TGF-β are hyperproliferative and expand markedly. After repeated stimulation one or two more time, however, they become anergic and respond poorly to T cell stimulants, but FOXP3 expression and suppressive activity by these cells is markedly greater than total T cells stimulated without azacytidine.

Remaining cells are restimulated with anti-CD3/anti-CD28 (at 1 in 10) with or without fresh azacytidine and/or azacytidine plus retinoic acid, such that there is a group from each CD4+ subset that is exposed to azacytidine/azacytidine+retinoic acid for the first time. After a further 6 days, the cells are assayed again for FOXP3 expression, suppressor activity and proliferative activity.

Both CD4+ cells and CD8+ cells express FOXP3 and demonstrate suppressive activity. In this example, Tregs suitable for T cell therapy are prepared without the need to purify specific T cell populations.

### Example 8

### Assessment of Cytokine Production

Cells from primary and secondary cultures are stimulated without any antigen presenting cells in serum free medium. The cells are stimulated with immobilized anti-CD3 or anti-CD3/anti-CD28 beads. Cytokine amounts are determined from supernatants collected on days 1 and 3 of culture using a cytokine bead array kit to measure IL-2, IFN, TNF, IL-6, IL-10 and IL-4. In addition, both active and latent TGF-β are measured using an ELISA kit. Cytokine amounts are determined from supernatants collected on days 1 and 3 of culture for measurement of IL-2, IL-4, IL-6, IFN-γ, and tumor necrosis factor (TNF) and at days 4 to 6 for IL-10 and both active and latent TGF-β using a cytokine bead array kit and a ELISA kits to measure the cytokines.

### Example 9

### Assessment of Methylation Status

Methylation status is initially determined using a procedure called COBRA (combined bisulfite restriction analysis). This method combines bisulfite treatment and PCR amplification of specific sites of the gene of interest. For human studies, the focus is on amplicon 5. Parallel studies are performed testing the ability of regulatory compositions comprising azacytidine to generate iTregs with the characteristics of nTreg using murine naïve CD4+ spleen cells.

### Example 10

### Assessment of Stability and Homeostatic Properties of Azacytidine-Generated iTregs

This assessment utilizes mice engineered to express a GFP-FOXP3 fusion protein. CD4+GFP-cells isolated by cell sorting are stimulated in the presence or absence of regulatory compositions containing azacytidine and regulatory compositions containing azacytidine and retinoic acid. Stimulation is also tested with such regulatory compositions with and without TGF-β. Stimulation in the presence of the regulatory compositions or TGF-β induces FOXP3 expression. Purified populations of FOXP3+ cells are isolated by cell sorting. As a positive control, nTregs are also sorted from fresh spleens and lymph nodes. Both iTregs and nTregs cells (5×10⁶) are injected into congenic CD45.1-mice. For the azacytidine-generated iTreg there are enough cells to have three to four mice per group. There are at least two mice per group for the nTregs. In some experiments, nTregs are expanded in culture to obtain sufficient numbers of cells. Prior to injection, the various populations are assayed for expression of chemokine/homing receptors such as CD103 (skin and gut), CD62L (lymph node) and CXCR4 (bone marrow).

Assessments are made at relatively early (day 7) and late (day 21) time points. The mice are sacrificed and the number of CD45.1+ cells that are FOXP3+ and FOXP3− cells are determined in various organs (blood, lymph node, spleen and bone marrow). It is determined if the cells generated with regulatory compositions containing azacytidine and/or retinoic acid (with or without TGF-β) behave similarly to the nTregs, which typically maintain their FOXP3 expression.

### Example 11

### Assessment of Ability of iTreg Generated Using Azacytidine to Beneficially Impact Autoimmune Disease

The K/B×N murine model of arthritis is used. The first step is the generation iTregs from histocompatible, non-transgenic C57BI/6×NOD (B×N) mice. Naïve CD4+ cells, at concentrations ranging from 1×10⁶ to 10×10⁶ are injected intravenously into 3 week or 5 week old K/B×N mice. Experiments with the 3 week old mice show the effect of iTregs on disease development, whereas experiments with the 5 week old mice show the effect of iTregs the established disease. The clinical severity of disease is scored as follows: 0, normal; 1, slight erythema and mild swelling confined to the mid-foot (tarsals) or ankle joint; 2, erythema and mild swelling extending from the ankle to the mid-foot; 3, erythema and moderate swelling extending from the ankle to the metatarsal joints and 4, intensive erythema and severe swelling encompassing ankle, foot and digits. All hind paws are graded, resulting in a maximal clinical score of 8 per mouse, and expressed asthe mean arthritic index on a given day. Mice are scored as arthritic if more than one paw has a score >2. The circumference of the ankle of each hind paw is measured with a caliper.

The effect of iTregs generated according to conventional methods (i.e., using regulatory compositions comprising TGF-β and optionally one or more other cytokines such as IL-2) in such mouse models are compared to the effect of iTregs generated using regulatory compositions described herein, including regulatory compositions comprising azacytidine and optionally retinoic acid and/or one or more cytokines, including TGF-β and IL-2.

Once azacytidine-generated iTregs from the B×N mice have shown therapeutic efficacy it is then determined if cells from the K/B×N mice can function similarly. Cells isolated from mice prior to, and subsequent to, arthritis development are tested for their ability to ameliorate disease. By isolating naïve CD4+ cells from mice with established disease, it is possible to mimic the situation that would exist if this procedure was to be used in a clinical setting.

In addition to mice treated with azacytidine-generated iTregs, the studies also include mice injected with fresh naïve CD4+ cells and mice injected with iTregs generated in the absence of azacytidine and retinoic acid. The reason for this comparison is to distinguish true regulatory effects from those attributable to an inhibition of homeostatic proliferation.

The above protocols may also be used to study the effect of regulatory compositions comprising azacytidine, TGF-β, IL-2, retinoic acid, trichostatin A, and combinations of two or more of these agents in animal models of collagen-induced arthritis.

The present specification provides a complete description of the methodologies, systems and/or structures and uses thereof in example aspects of the presently-described technology. Although various aspects of this technology have been described above with a certain degree of particularity, or with reference to one or more individual aspects, those skilled in the art could make numerous alterations to the disclosed aspects without departing from the spirit or scope of the technology hereof. Since many aspects can be made without departing from the spirit and scope of the presently described technology, the appropriate scope resides in the claims hereinafter appended. Other aspects are therefore contemplated. Furthermore, it should be understood that any operations may be performed in any order, unless explicitly claimed otherwise or a specific order is inherently necessitated by the claim language. It is intended that all matter contained in the above description and shown in the accompanying drawings shall be interpreted as illustrative only of particular aspects and are not limiting to the embodiments shown. Unless otherwise clear from the context or expressly stated, any concentration values provided herein are generally given in terms of admixture values or percentages without regard to any conversion that occurs upon or following addition of the particular component of the mixture. To the extent not already expressly incorporated herein, all published references and patent documents referred to in this disclosure are incorporated herein by reference in their entirety for all purposes. Changes in detail or structure may be made without departing from the basic elements of the present technology as defined in the following claims.

What is claimed:

1. A method of generating regulatory T cells (Tregs), the method comprising treating a cell culture comprising non-regulatory T cells with a regulatory composition comprising an agent that prevents methylation of a gene encoding a transcription factor.

2. The method of claim 1, wherein the agent is a methyltransferase inhibitor.

3. The method of claim 2, wherein the methyltransferase inhibitor is azacytidine.

4. The method of claim 2, wherein the regulatory composition further comprises a cytokine.

5. The method of claim 4, wherein the cytokine is TGF-β.

6. The method of claim 2, wherein the regulatory composition further comprises an agent that accelerates T cell differentiation.

7. The method of claim 6, wherein the agent that accelerates T cell differentiation is retinoic acid.

8. The method of claim 2, wherein the regulatory composition further comprises an agent that is a histone deacetylase inhibitor.

9. The method of claim 8, wherein the histone deacetylase inhibitor is trichostatin A.

10. The method of claim 1, wherein the treating of the cell culture comprises adding the regulatory composition at the initiation of the culture.

11. The method of claim 10, further comprising adding an agent to the cell culture at a second time point subsequent to the addition of the regulatory composition at the initiation of the culture.

12. The method of claim 11, wherein the agent is a member selected from: azacytidine, retinoic acid, trichostatin A, TGF-β, IL-2, anti-CD3, anti-CD28, and a combination thereof.

13. The method of claim 1, wherein the treating of the cell culture comprises adding the regulatory composition after initiation of the culture.

14. The method of claim 1, wherein the cell culture is maintained for about one week after the treating with the regulatory composition.

15. A method of treating an aberrant immune response or an autoimmune disease in a patient, the method comprising administering regulatory T cells to the patient, wherein the regulatory T cells are generated by treatment of a cell culture comprising non-regulatory T cells with a regulatory composition comprising a member selected from: azacytidine, retinoic acid, trichostatin A, and a combination thereof.

16. The method of claim 15, wherein the regulatory composition further comprises TGF-β.

17. The method of claim 16, wherein the regulatory composition further comprises IL-2.

18. The method of claim 17, wherein the regulatory composition further comprises a T cell activator.

19. The method of claim 15, wherein the cell culture is stimulated with a T cell activator prior to, simultaneously with, or subsequent to the treatment with the regulatory composition.

20. The method of claim 19, wherein the T cell activator is anti-CD3, anti-CD28, or a combination of anti-CD3 and anti-CD28.

21. A method of generating regulatory T cells (Tregs), the method comprising treating a cell culture comprising non-regulatory T cells with a regulatory composition comprising an agent that accelerates differentiation of T cells into Tregs.

22. The method of claim 21, wherein the agent that accelerates differentiation of T cells comprises retinoic acid.

23. The method of claim 22, wherein the regulatory composition further comprises a member selected from IL-2, TGF-β, and a combination of IL-2 and TGF-β.

24. The method of claim 23, wherein the regulatory composition further comprises a methyltransferase inhibitor.

25. The method of claim 24, wherein the regulatory composition further comprises a histone deacetylase inhibitor.

26. The method of claim 25, wherein the methyltransferase inhibitor is azacytidine and the histone deacetylase inhibitor is a member selected from trichostatin A and retinoic acid.

27. The method of claim 18, wherein the regulatory composition further comprises at least one T cell activator.

28. A composition comprising:
a. cell culture medium;
b. azacytidine;
c. retinoic acid;
d. a population of T cells, wherein the population T cells comprises at least one naïve CD4+ cell.

29. The composition of claim 20, wherein the population of T cells further comprises at least one induced regulatory T cell.

30. The composition of claim 21, wherein the at least one induced regulatory T cell is a suppressor T cell.

31. The composition of claim 28, further comprising trichostatin A.

32. A kit comprising:
a. a regulatory composition comprising a member selected from: azacytidine, retinoic acid, and a combination of azacytidine and retinoic acid;
b. a cell treatment container;
c. written instructions for use of the kit.

33. The kit of claim 32, wherein the regulatory composition further comprises TGF-β, IL-2, or a combination of TGF-β and IL-2.

34. The kit of claim 33, wherein the regulatory composition further comprises trichostatin A.

35. The kit of claim 32, wherein the cell treatment container comprises a port adapted for attachment to a leukopheresis machine.
